(19) United States
(12) Patent Application Publication
(10) Pub. No.: US 2016/0304910 A1
(43) Pub. Date: Oct. 20, 2016

(54) RECOMBINANT STRAIN OF SACCHAROMYCES CEREVISIAE OVERPRODUCING GLYCEROL

(71) Applicant: ARCHER DANIELS MIDLAND COMPANY, Decatur, IL (US)

(72) Inventors: Andriy Sibirny, Lviv (UA); Kostyantyn V Dmytruk, Lviv (UA); Charles Abbas, Champaign, IL (US); Lidiia R. Murashchenko, Lviv (UA)

(21) Appl. No.: 15/101,966

(22) PCT Filed: Dec. 5, 2014
(86) PCT No.: PCT/US14/68699

§ 371 Date: Jun. 6, 2016

Related U.S. Application Data
(60) Provisional application No. 61/915,252, filed on Dec. 12, 2013.

(51) Int. Cl. C12P 7/20; C12N 9/10

(52) U.S. Cl. CPC C12P 7/20; C12N 9/1022; C12Y 202/01006; C12N 2800/102

(57) ABSTRACT

A truncated version of Saccharomyces cerevisiae IVL2 gene encoding a cytosolic form of acetolactate synthase was cloned into an expression cassette under control of a strong constitutive alcohol dehydrogenase (ADH1) promoter. The plasmid was introduced into the S. cerevisiae strain and the recombinant strain was tested for ability to overproduce glycerol under anaerobic conditions. It was shown that the recombinant strain was characterized by increased glycerol production and decreased ethanol production under anaerobic conditions.

### CROSS-REFERENCE TO RELATED APPLICATIONS

This application claims priority to U.S. Provisional Application 61/915,252 filed Dec. 12, 2013.

### FIELD OF THE INVENTION

The present disclosure relates to modified yeast for enhanced production of glycerol, more particularly to yeast modified to overexpress a truncated ILV2 gene in S. cerevisiae, resulting in an increase in glycerol production under anaerobic growth conditions.

### BACKGROUND OF THE INVENTION

Glycerol (1, 2, 3-propanetriol) is used in the cosmetic, paint, automotive, food, tobacco, pharmaceutical, pulp and paper, leather and textile industries. Glycerol has primarily been recovered as a by-product of biodiesel or soap manufacturing or produced from propylene and allyl alcohol. Alternatively, glycerol has also been produced by microbial fermentation, using carbohydrate-based feedstocks. Glycerol has also been considered as a feedstock for new industrial fermentations. For example, glycerol has been fermented to 1, 3-propanediol, dihydroxyacetone and dihydroxyacetone phosphate and other value-added products.

Due to environmental concerns, the chemical synthesis of glycerol from propylene or allyl alcohol is on the decline. The glycerol obtained as a by-product from biodiesel or soap production contains a lot of impurities, which precludes its further use without additional refining.

Several processes are known for the microbial synthesis of glycerol from carbohydrates that use osmotolerant yeasts, algae and a number of bacteria. However, all these utilize aerobic conditions, so the glycerol production demands air or oxygen purging throughout the fermentation, which considerably increases production costs.

It is known that wild-type strains of S. cerevisiae are able to produce substantial amounts of glycerol after adding sulfites to the fermentation medium, which results in trapping of acetaldehyde at an alkaline pH. However, glycerol yield from this sulfite process is typically low, and this mode of production results in large buildup of sulfite, leading to waste stream byproduct disposal problems. The metabolic engineering of S. cerevisiae strains with high yield of glycerol that accumulates under anaerobic conditions without buildup of waste byproducts would be much more efficient. However, whereas anaerobic glucose conversion to ethanol is energy positive (net yield of 2 ATP per mole of glucose), anaerobic glycerol production from glucose presents a major challenge as it is an energy-negative process (minus 2 moles of ATP per mole of glucose). This is because glucose first has to be phosphorylated twice, with no production of ATP during glycerol synthesis. This means that yeast cannot survive anaerobically when it accumulates only glycerol, as it needs energy for cell maintenance. To be able to survive, cells either need to get some oxygen for ATP production by oxidative phosphorylation, or need to accumulate some by-product, the synthesis of which is coupled to ATP production. If the cells accumulate equal amounts of glycerol and ethanol, ATP balance is zero and this ratio of glycerol to ethanol can theoretically support strict anaerobic growth. Demonstration of this hypothesis can be demonstrated through the construction of yeast strains through the use of genetic engineering that allow for the production of glycerol and ethanol under anaerobic conditions where the strains can derive sufficient ATP and NADH.

There are a number of publications that describe the increase in glycerol production by S. cerevisiae mutants deleted in one or several ADH genes coding for alcohol dehydrogenases (Drewke et al., 1990) or PDC1 and PDC5 genes coding for pyruvate decarboxylases (Nevoigt and Stahl, 1996). Mutants defective in alcohol dehydrogenase typically accumulate large amounts of acetaldehyde and acetic acid which are toxic to yeast cells. Mutants defective in pyruvate decarboxylase look more promising. However, they cannot grow without adding exogenous ethanol or acetate, as pyruvate decarboxylase is the only source of two-carbon compounds in cytosol that are needed mainly for lipid biosynthesis (Pronk et al., 1996). The largest amount of glycerol is accumulated by S. cerevisiae mutants deleted in the TPI1 gene coding for triose phosphate isomerase (Overkamp et al., 2002). Unfortunately, such mutants fail to grow on glucose as the primary carbon source. Metabolic engineering of an alternative NADH reoxidation pathway (isocitrate lyase) has allowed partial restoration of the growth on glucose, however the robustness of strains was low. Additionally, glucose inhibited growth of the analyzed TPI1 strains.

Therefore, there is continuing need in the field to develop yeast that are capable of efficient glycerol production from glucose under anaerobic conditions.

### BRIEF SUMMARY OF THE INVENTION

The present disclosure provides methods for increasing glycerol production by increasing expression of acetolactate synthase in recombinant yeast and genetic tools for producing said recombinant yeasts. One embodiment of the disclosure comprises recombinant nucleic acid with a truncated portion of a gene encoding a cytosol located acetolactate synthase activity, wherein said gene is operably linked to a non-native promoter to express said acetolactate synthase activity in the cytosol.

A further embodiment comprises a nucleic acid molecule with a gene encoding the protein of a cytosol located acetolactate synthase, wherein said gene is at least 75% identical to SEQ ID NO:2 and does not contain a mitochondrial targeting signal.

A further embodiment includes the recombinant nucleic acid molecule with the cytosol located acetolactate synthase activity is gene is a SEQ ID NO: 1.

One embodiment of the disclosure contains the recombinant nucleic acid molecule with an ADH1 promoter operably linked to the truncated ILV2 gene. A further embodiment comprises a vector containing the truncated ILV2 gene operably linked to a strong constructive promoter. An additional embodiment comprises the vector including a selective marker. An even further embodiment contains the selective marker of the vector being a natNT2 gene.

One embodiment of the disclosure is a host cell includes a vector containing the truncated ILV2 gene operably linked to a strong constructive promoter. An additional embodiment comprises a host cell comprising the vector containing the truncated ILV2 gene operably linked to a strong constructive promoter and including a selective marker. A further embodiment comprises the host cell is S. cerevisiae cell.

One embodiment of the disclosure is a yeast strain containing a recombinant nucleic acid molecule comprising a truncated portion of a gene encoding a cytosol located acetolactate synthase activity that is operably linked to a non-native promoter to express the acetolactate synthase activity in the cytosol.

A further embodiment includes a yeast strain with a truncated gene that does not comprise a mitochondrial targeting signal. A further embodiment includes a yeast strain comprising a truncated portion of a gene encoding a cytosol located acetolactate synthase activity that is operably linked to a non-native promoter to express the acetolactate synthase activity in the cytosol when the truncated gene is according to SEQ ID NO: 1. A further embodiment is when promoter element of this yeast strain is an ADH1 promoter. An even further embodiment is when this yeast strain is S. cerevisiae.

One embodiment of this disclosure includes a method for enhancing glycerol production in yeast comprising growing the yeast strain comprising a recombinant nucleic acid molecule comprising a truncated portion of a gene encoding a cytosol located acetolactate synthase activity that is operably linked to a non-native promoter to express the acetolactate synthase activity in the cytosol anaerobically in a culture medium, under conditions that cause the yeast strain to make glycerol.

A further embodiment includes a method of growing the yeast strain comprising a recombinant nucleic acid molecule comprising a truncated portion of a gene encoding a cytosol located acetolactate synthase activity that is operably linked to a non-native promoter to express the acetolactate synthase activity in the cytosol anaerobically in a culture medium, under conditions that cause the yeast strain to make glycerol, wherein an amount of glycerol produced by said yeast strain is more than four times greater than an amount of glycerol produced by a corresponding yeast strain that does not contain said recombinant nucleic acid molecule but is otherwise identical to said yeast strain.

A further embodiment includes the yeast strain to be S. cerevisiae.

### BRIEF DESCRIPTION OF THE DRAWINGS

FIG. 1 shows a linear scheme of recombinant plasmid pUC57-ADHI-ILV2-natNT2 (A) and a linear scheme of recombinant plasmid pUC57-natNT2-TPI1-ILV2n (B).

FIG. 2 shows the complete sequence of recombinant plasmid pUC57-ADHI-ILV2-natNT2. Components of complete sequence are represented by font style and bold differences, as follows: pUC57 in Calibri 11 (CGGAT), ADHI in Calibri 8 (CGGAT), ILV2 in Calibri 11 Bold (CGGAT), natNT2 in Tahoma 12 Italic (CGGAT), loxP in Tahoma 12 Bold (CGGAT)

FIG. 3 shows the complete sequence of recombinant plasmid pUC57-natNT2-TPI1-ILV2n. Components of complete sequences are represented by font style and bold differences as follows: pUC57 in Calibri 11 (CGGAT), TPI1 in Calibri 8 (CGGAT); ILV2 in Calibri 11 Bold (CGGAT), natNT2 in Tahoma 12 Italic (CGGAT).

FIG. 4 shows yeast metabolism of glucose and catalytic action of acetolactate synthase to yield glycerol and ethanol.

### SEQUENCE LISTING

SEQ ID NO: 1—The nucleic acid sequence of the truncated ilV2 gene from S. cerevisiae.

SEQ ID NO: 2—The amino acid sequence of the truncated ilV2 gene from S. cerevisiae.

SEQ ID NO: 3—The nucleic acid sequence of the full length native ilV2 gene from S. cerevisiae.

SEQ ID NO: 4—The amino acid sequence of the full length native ilV2 gene from S. cerevisiae.

SEQ ID NO: 5—The nucleic acid sequence of the ADH1 promoter

SEQ ID NO: 6—The nucleic acid sequence of the TPI1 promoter

SEQ ID NO: 7—The nucleic acid sequence of the natNT2 marker

SEQ ID NO: 8—The nucleic acid sequence of loxP, used to facilitate marker rescue

SEQ ID NO: 9—The nucleic acid sequence of pUC57-ADHI-ILV2-natNT2

SEQ ID NO: 10—The nucleic acid sequence of pUC57-natNT2-TPI1-ILV2n

SEQ ID NO: 11—Ko574 Primer used to PCR amplify truncated ILV2

SEQ ID NO: 12—Ko575 Primer used to PCR amplify truncated ILV2

SEQ ID NO: 13—Ko572 Primer used to PCR amplify ADH1 promoter

SEQ ID NO: 14—Ko573 Primer used to PCR amplify ADH1 promoter

SEQ ID NO: 15—OK19 Primer used to PCR amplify natNT2 marker for cloning into plasmid pUC57-ADHI-ILV2-natNT2

SEQ ID NO: 16—OK20 Primer used to PCR amplify natNT2 marker for cloning into plasmid pUC57-ADHI-ILV2-natNT2

SEQ ID NO: 17—LY7 Primer used to PCR amplify TPI1 promoter

SEQ ID NO: 18—LY8 Primer used to PCR amplify TPI1 promoter

SEQ ID NO: 19—LY9 primer to amplify the full length native ilV2 gene from S. cerevisiae.

SEQ ID NO: 20—LY10 primer to amplify the full length native ilV2 gene from S. cerevisiae.

SEQ ID NO: 21—Ko446 Primer used to amplify natNT2 for cloning into pUC57 natNT2-TPI1-ILV2n

SEQ ID NO: 22—Ko448 Primer used to amplify natNT2 for cloning into pUC57 natNT2-TPI1-ILV2n

SEQ ID NO: 23—full length ILV2 gene from Saccharomyces kudriavzevil (The truncated version of this gene (amino acid 56-687) has 94.5% homology to the truncated ILV2 from S. cerevisiae=SEQ ID NO: 2.)

SEQ ID NO: 24—full length ILV2 gene from Naumovozyma castellii (The truncated version of this gene (amino acid 52-684) has 85.2% homology to the truncated ILV2 from S. cerevisiae=SEQ ID NO: 2.)

SEQ ID NO: 25—full length ILV2 gene from Naumovozyma dairenensis (The truncated version of this gene (amino acid 54-685) has 83.4% homology to the truncated ILV2 from S. cerevisiae=EQ ID NO: 2.)

SEQ ID NO: 26—full length ILV2 gene from Candida glabrata (The truncated version of this gene (amino acid 41-677) has 82.8% homology to the truncated ILV2 from S. cerevisiae=SEQ ID NO: 2.)

SEQ ID NO: 27—full length ILV2 gene from Torulaspora delbrueckii (The truncated version of this gene (amino acid 53-682) has 80.9% homology to the truncated ILV2 from S. cerevisiae=SEQ ID NO: 2.)

SEQ ID NO: 28—full length ILV2 gene from Kazachstania africana (The truncated version of this gene (amino acid 51-682) has 80.8% homology to the truncated ILV2 from S. cerevisiae=SEQ ID NO: 2.)

SEQ ID NO: 29—full length ILV2 gene from Kazachstania naganishii (The truncated version of this gene (amino acid 53-693) has 80.6% homology to the truncated ILV2 from S. cerevisiae=SEQ ID NO: 2.)

SEQ ID NO: 30—full length ILV2 gene from Vanderwaltozyma polyspora (The truncated version of this gene (amino acid 46-671) has 79.8% homology to the truncated ILV2 from S. cerevisiae=SEQ ID NO: 2.)

SEQ ID NO: 31—full length ILV2 gene from Zygosaccharomyces rouxii (The truncated version of this gene (amino acid 72-700) has 79.2% homology to the truncated ILV2 from S. cerevisiae=SEQ ID NO: 2.)

SEQ ID NO: 32—full length ILV2 gene from Zygosaccharomyces bailii (The truncated version of this gene (amino acid 55-685) has 78.5% homology to the truncated ILV2 from S. cerevisiae=SEQ ID NO: 2.)

SEQ ID NO: 33—full length ILV2 gene from Kluyveromyces lactis (The truncated version of this gene (amino acid 46-691) has 77.7% homology to the truncated ILV2 from S. cerevisiae=SEQ ID NO: 2.)

SEQ ID NO: 34—full length ILV2 gene from Kluyveromyces marxianus (The truncated version of this gene (amino acid 68-700) has 77.25% homology to the truncated ILV2 from S. cerevisiae=SEQ ID NO: 2.)

SEQ ID NO: 35—full length ILV2 gene from Lachancea thermotolerans (The truncated version of this gene (amino acid 49-678) has 76.8% homology to the truncated ILV2 from S. cerevisiae=SEQ ID NO: 2.)

SEQ ID NO: 36—full length ILV2 gene from Tetrapisispora phaffia (The truncated version of this gene (amino acid 53-688) has 76.6% homology to the truncated ILV2 from S. cerevisiae=SEQ ID NO: 2.)

SEQ ID NO: 37—full length ILV2 gene from Tetrapisispora blattae (The truncated version of this gene (amino acid 79-710) has 75.4% homology to the truncated ILV2 from S. cerevisiae=SEQ ID NO: 2.)

### DETAILED DESCRIPTION OF PREFERRED EMBODIMENTS

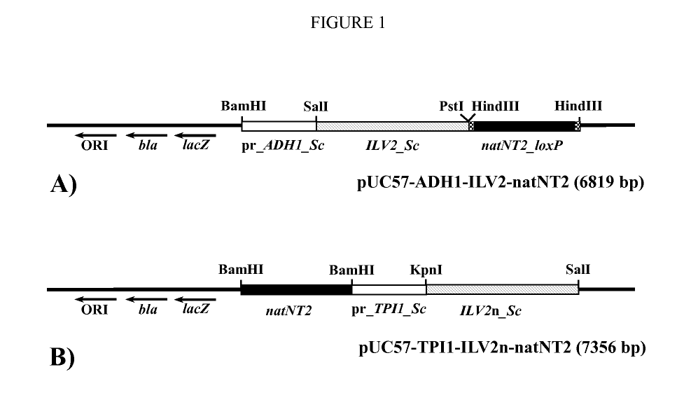

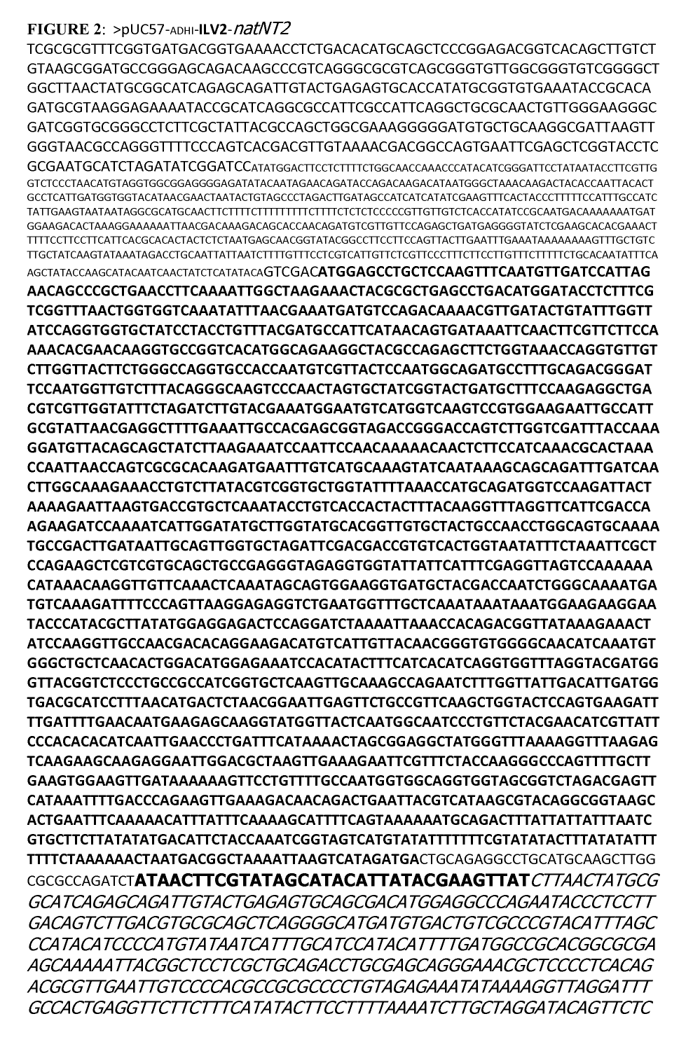

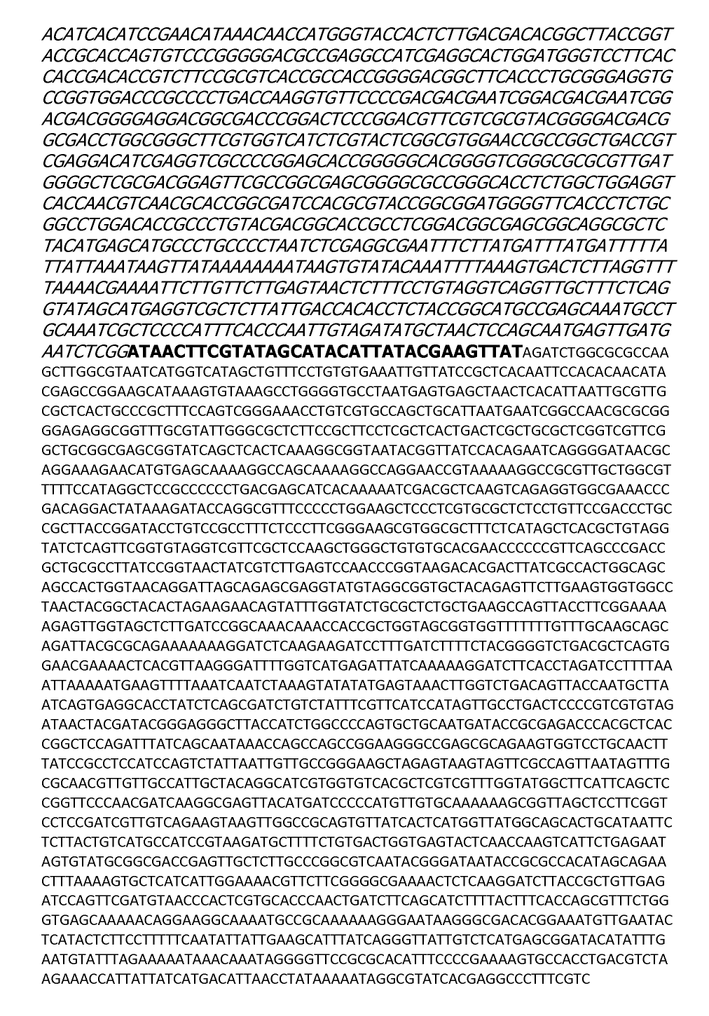

It has been discovered that overexpression of the truncated version (deficient 5′-165 by and lacking a mitochondrial targeting signal) (SEQ ID NO: 1) of the yeast ILV2 gene, encoding for acetolactate synthase, strongly activates glycerol production under anaerobic conditions. One aspect of the present invention is directed to a recombinant nucleic acid molecule formed by fusing the truncated ILV2 gene with a strong constitutive promoter element. The source of the ILV2 gene could be selected from, but not limited to, a eukaryotic microorganism. One suitable promoter element is the promoter of gene ADH1 encoding alcohol dehydrogenase. The truncated ILV2 gene, operably linked to the promoter element, is cloned into a vector. Plasmid pUC57 is one suitable vector. Preferably a selective marker is also cloned into the plasmid. Dominant selective marker natNT2, conferring resistance to antibiotic nourseothricin (NTC) for selection in yeast, is one suitable selection marker. One embodiment of the invention is directed to recombinant plasmid pUC57-ADHI-ILV2-natNT2 (SEQ ID NO: 9) (FIG. 1 (A): FIG. 2 shows the DNA sequence) harboring an expression cassette for overexpression of the truncated version of the S. cerevisiae ILV2 gene.

The recombinant nucleic acid molecule comprising the truncated ILV2 gene, operably linked to a promoter, is transformed into a host cell. In one preferred embodiment, a Saccharomyces cerevisiae strain is used as the host strain, more preferably, S. cerevisiae strain BY4742.

As discussed in more detail below, the novel recombinant strain BY4742/ILV2 of S. cerevisiae, when grown anaerobically in a suitable medium, under conditions that cause the yeast strain to make glycerol, was shown to produce as much as 4 g of glycerol/L, which was a 4.4-fold improvement in glycerol production as compared to parental strain BY4742.

In a preferred embodiment, a nucleic acid molecule encoding the truncated ILV2 gene encoding a protein sequence would be at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 2.

The novel recombinant strain BY4742/ILV2 of S. cerevisiae expresses a presumably cytosolic form of ILV2. While not wanting to be bound by any particular mode of increased glycerol production, the following is offered as a possible mode. The acetolactate synthase encoded by ILV2 is normally transported into the mitochondria where it is sequestered from the cytosolic pool of NAD(H) and pyruvate that is used during anaerobic fermentation. The activity of the enzyme is to catalyze the condensation of two pyruvate molecules into an acetolactate molecule with release carbon dioxide. We suggest that with the transit peptide removed, the enzyme activity is retained in the cytosol where it reduces the available pyruvate pool that ordinarily would proceed predominantly to ethanol and acetate production under anaerobic conditions. The last step in ethanol production is the conversion of acetaldehyde to ethanol by alcohol dehydrogenase with oxidation of NADH to NAD+, which depletes the NADH pool. Because less pyruvate is available for conversion to ethanol, less cytosolic NADH is consumed thereby allowing more of the NADH to be used upstream by NADH dependent glycerol 3-phosphate dehydrogenase, which in turns leads to increased production of glycerol-3-phosphate and its subsequent dephosphoryalation to glycerol (FIG. 2). Unlike the case where ADH activity is completely eliminated, there is still reduction in toxic levels of acetaldehyde produced by the action of alcohol dehydgrogenase. Since pyruvate decarboxylase is not eliminated, there still remains adequate production of cytosolic acetate from pyruvate, with the acetate being necessary for fatty acid synthesis. Accordingly, the increased expression of cytosolic acetolactate synthase is believed to reduce, but not eliminate, the cytosolic pool of pyruvate, leaving sufficient levels of this important metabolic intermediate to maintain fatty acid synthesis. This also permits increased flow of the phosphorylated 3 carbon metabolite into glycerol production due to higher NADH availability because not as much pyruvate is being shunted to ethanol via alcohol dehydrogenase.

### A. DEFINITIONS

The term “gene” refers to a DNA sequence that comprises coding sequences and optionally control sequences necessary for the production of a polypeptide from the DNA sequence.

The term “recombinant DNA” molecule means a hybrid DNA sequence comprising at least two nucleotide sequences not normally found together in nature.

The term “vector” is used in reference to nucleic acid molecules into which fragments of DNA may be inserted or cloned and can be used to transfer DNA segments into a cell and capable of replication in a cell. Vectors may be derived from plasmids, bacteriophages, viruses, cosmids, and the like.

The terms “recombinant vector”, “expression vector” or “construct” as used herein refer to DNA or RNA sequences containing a desired coding sequence and appropriate DNA or RNA sequences necessary for the expression of the operably linked coding sequence in a particular host organism. Prokaryotic expression vectors include a promoter, a ribosome binding site, an origin of replication for autonomous replication in a host cell and possibly other sequences, e.g. an optional operator sequence, optional restriction enzyme sites. A promoter is defined as a DNA sequence that directs RNA polymerase to bind to DNA and to initiate RNA synthesis. Eukaryotic expression vectors include a promoter, optionally a polyadenylation signal and optionally an enhancer sequence.

A polynucleotide having a nucleotide sequence “encoding a peptide, protein or polypeptide” means a nucleic acid sequence comprising a coding region for the peptide, protein or polypeptide. The coding region may be present in either a cDNA, genomic DNA or RNA form. When present in a DNA form, the oligonucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region, utilized in the expression vectors of the present invention may contain endogenous enhancers/promoters, splice junctions, intervening sequences, polyadenylation signals, etc. In further embodiments, the coding region may contain a combination of both endogenous and exogenous control elements.

Promoters and enhancers consist of short arrays of DNA sequences that interact specifically with cellular proteins involved in transcription. Promoter and enhancer elements have been isolated from a variety of eukaryotic sources including genes from yeast, insect and mammalian cells. Promoter and enhancer elements have also been isolated from viruses and analogous control elements, such as promoters, are also found in prokaryotes. The selection of a particular promoter and enhancer depends on the cell type used to express the protein of interest. The enhancer/promoter may be “endogenous” or “exogenous” or “heterologous.” An “endogenous” enhancer/promoter is one that is naturally linked with a given gene in the genome. An “exogenous” or “heterologous” enhancer/promoter is one that is placed in juxtaposition to a gene by means of genetic manipulation (i.e., molecular biological techniques) such that transcription of the gene is directed by the linked enhancer/promoter. A “constitutive promoter” is an unregulated promoter that allows for continual transcription of its associated gene.

The term “expression system” refers to any assay or system for determining (e.g., detecting) the expression of a gene of interest. Those skilled in the field of molecular biology will understand that any of a wide variety of expression systems may be used.

The term “recombinant protein” or “recombinant polypeptide” as used herein refers to a protein molecule expressed from a recombinant DNA molecule. In contrast, the term “native protein” is used herein to indicate a protein isolated from a naturally occurring (i.e., a non-recombinant or wild type) source. Molecular biological techniques may be used to produce a recombinant form of a protein with identical properties as compared to the native form of the protein.

The terms “cell,” “cell line,” “host cell,” as used herein, are used interchangeably, and all such designations include progeny or potential progeny of these designations. By “transformed cell” is meant a cell into which (or into an ancestor of which) has been introduced a nucleic acid molecule of the invention. Optionally, a nucleic acid molecule of the invention may be introduced into a suitable cell line so as to create a stably transfected cell line capable of producing the protein or polypeptide encoded by the nucleic acid molecule. Vectors, cells, and methods for constructing such cell lines are well known in the art. The words “transformants” or “transformed cells” include the primary transformed cells derived from the originally transformed cell without regard to the number of transfers. All progeny may not be precisely identical in DNA content, due to deliberate or inadvertent mutations. Nonetheless, mutant progeny that have the same functionality as screened for in the originally transformed cell are included in the definition of transformants.

The term “operably linked” as used, herein refer to the linkage of nucleic acid sequences in such a manner that a nucleic acid molecule capable of directing the transcription of a given gene and/or the synthesis of a desired protein molecule is produced. The term also refers to the linkage of sequences encoding amino acids in such a manner that a functional (e.g., enzymatically active, capable of binding to a binding partner, capable of inhibiting, etc.) protein of polypeptide, or a precursor thereof, e.g., the pre- or prepro-form of the protein or polypeptide, is produced.

### B. MATERIALS AND METHODS USED FOR EXEMPLARY EMBODIMENTS

### Strains.

The S. cerevisiae strain BY4742 (MAT.alpha., his3.DELTA.1, leu2.DELTA.0, lys2.DELTA.0, ura3.DELTA.0; Giaever et al., 2002) was used for overexpression of the truncated version of the ILV2 gene. Escherichia coli DH5α strain Φ80dlacZΔM15, recA1, endA1, gyrA96, thi-1, hsdR17(r_{K}_{^{−}}, m_{K}_{⁺}), supE44, relA1, deoR, Δ(lacZYA-argF)U169 was used for subcloning.

### DNA Manipulation

Genomic DNA from S. cerevisiae was isolated using Wizard® Genomic DNA Purification Kit (Promega, Madison, Wis., USA). Plasmid DNA from E. coli was isolated using Wizard® Plus SV Minipreps DNA Purification System (Promega) and High Fidelity PCR Enzyme Mix and restriction enzymes were used according to recommendation of supplier (Thermo scientific, Vilnius, Lithuania, EU). S. cerevisiae transformation was performed by Sambrook and Russell 2001.

### Construction of Plasmid for Overexpression ILV2

Construction of the expression cassette pUC57-ADHI-ILV2-natNT2 for overexpression of S. cerevisiae gene ILV2 encoding acetolactate synthase, lacking a mitochondrial targeting signal, was performed using the ILV2 gene deficient in 5′-165 by (SEQ ID NO: 1) and a strong constitutive promoter of gene ADH1 encoding alcohol dehydrogenase. The truncated ILV2 gene and promoter were PCR-amplified using pairs of primers Ko574 (CAA TCA ACT ATC TCA TAT ACA GTC GAC ATG GAG CCT GCT CCA AGT TTC AA) (SEQ ID NO: 11)/Ko575 (AAA CTG CAG TCA TCT ATG ACT TAA TTT TAG CC) (SEQ ID NO: 12) and Ko572 (CGC GGA TCC ATA TGG ACT TCC TCT TTT CTG) (SEQ ID NO: 13) and Ko573 (TTG AAA CTT GGA GCA GGC TCC ATG TCG ACT GTA TAT GAG ATA GTT GAT TG) (SEQ ID NO: 14). Both fragments were combined via overlap PCR using primers Ko575 and Ko572. The fused DNA fragment harboring ADH1 promoter and truncated version of ILV2 gene was BamHI/PstI double digested and cloned to the BamHI/PstI linearized pUC57 plasmid cloning vector. The resulting plasmid was designated as pUC57-ADHI-ILV2.

A second marker gene natNT2, from Streptomyces noursei which provides for resistance to the antibiotic nourseothricin, was PCR-amplified using primer pair OK19 (CCC AAG CTT GGC GCG CCA GAT CTA TAA CTT CGT ATA GCA TAC ATT ATA CGA AGT TAT CTT AAC TAT GCG GCA TCA GAG) (SEQ ID NO: 15) and OK20 (CCC AAG CTT GGC GCG CCA GAT CTA TAA CTT CGT ATA ATG TAT GCT ATA CGA AGT TAT CCG AGA TTC ATC AAC TCA TTG C) (SEQ ID NO: 16), and used with plasmid pRS41N (Taxis and Knop 2006) as a template.

This 1348 by DNA fragment was HindIll digested and cloned to HindIII-linearized plasmid pUC57-ADHI-ILV2. The plasmid was transformed into E. coli as described above, and selection of E. coli transformants was performed on LB medium supplemented with nourseothricin at a concentration of 50 μg/mL. The constructed and verified plasmid was designated as pUC57-ADHI-ILV2-natNT2 (SEQ ID NO:9).

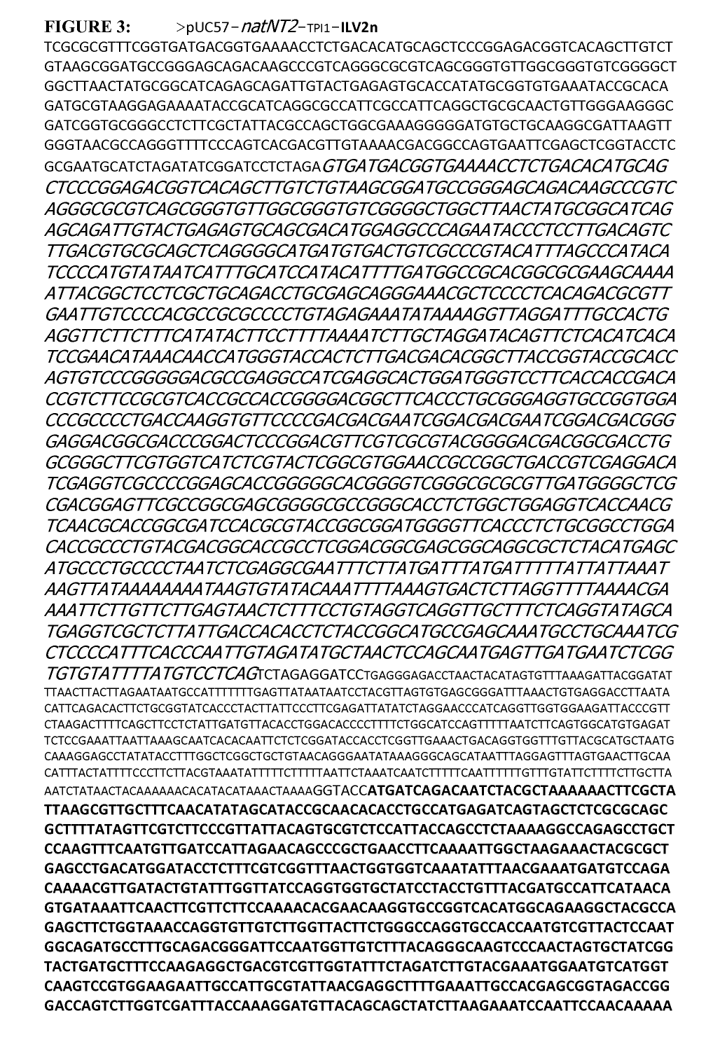

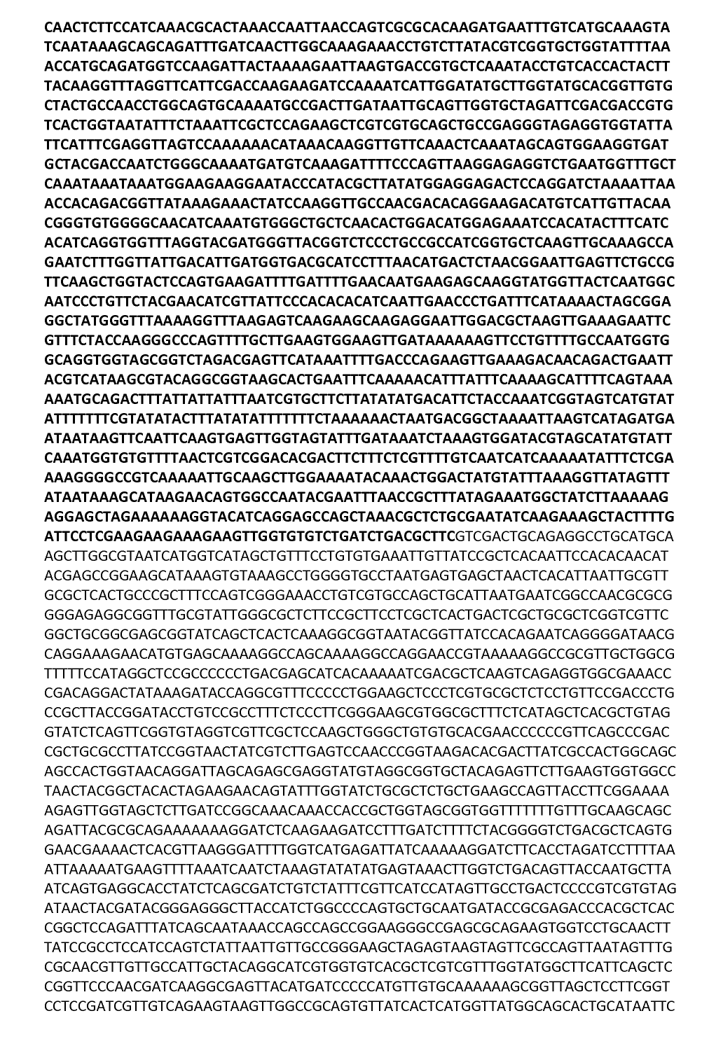

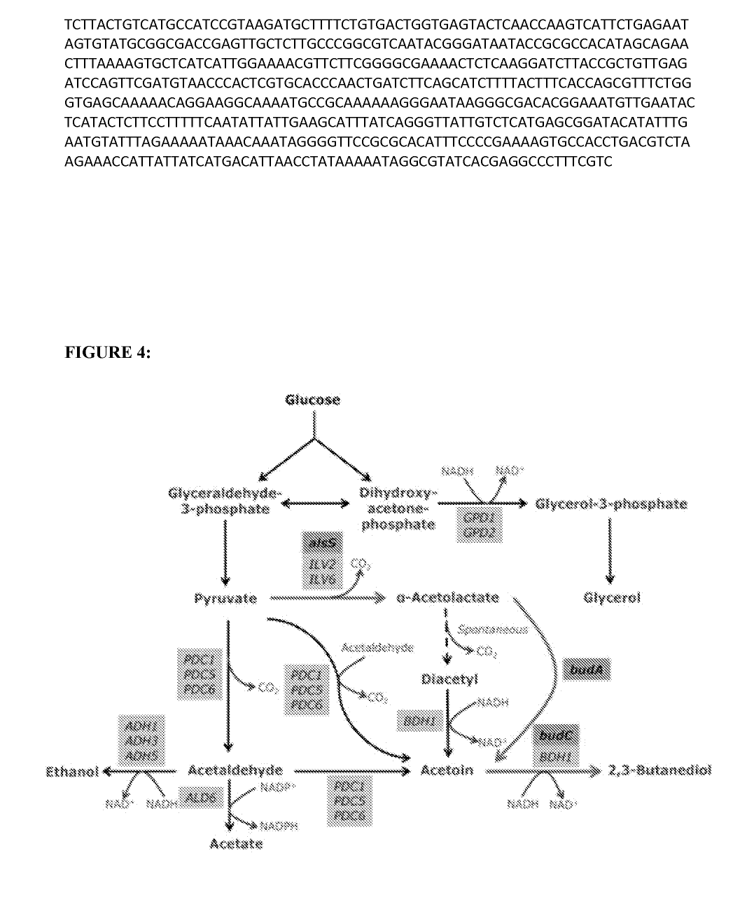

In order to overexpress the native ILV2 gene coding for the acetolactate synthase, this target gene was fused with the strong constitutive promoter of gene TPI1 encoding triose phosphate isomerase. The native mitochondrial Ilv2 protein catalyzes the first common step in isoleucine and valine biosynthesis pathway. The TPI1 promoter and ORF of ILV2 of S. cerevisiae in part with terminator sequence were amplified using pairs of primers LY7 (CGG GAT CCT GAG GGA GAC CTA ACT ACA TAG) (SEQ ID NO:17)/LY8 (TAG CGT AGA TTG TCT GAT CAT GGT ACC TTT TAG TTT ATG TAT GTG TTT TTT) (SEQ ID NO:18) and LY9 (AAA AAA CAC ATA CAT AAA CTA AAA GGT ACC ATG ATC AGA CAA TCT ACG CTA) (SEQ ID NO: 19)/LY10 (GCG TCG ACG AAG CGT CAG ATC AGA CAC A) (SEQ ID NO: 20) . Both fragments were combined using primers LY7/LY10 by overleap PCR and subsequently cloned to the BamHI/SalI double-digested pUC57 plasmid. This plasmid was designated pUC57-TPII-ILV2n. The gene natNT2 conferring resistance against the aminoglycoside antibiotic nourseothricin was amplified using primers Ko446 (CCG GGA TCC TCT AGA GTG ATG ACG GTG AAA ACC TCT G) (SEQ ID NO: 21)/Ko448 (CCG GGA TCC TCT AGA CTG AGG ACA TAA AAT ACA CAC CG) (SEQ ID NO: 22) and plasmid pRS41N as a template. The amplified fragment was digested with BamHI and ligated with BamHI digested and dephosphorylated plasmids pUC57-TPI1-ILV2n. The selection of E. coli transformants was performed as described above. Constructed and verified plasmid was designated as pUC57-natNT2-TPI1-ILV2n (FIG. 1 (B), DNA sequence in FIG. 3).

### Transformation, Selection and Characterization of Stable Transformants of S. Cerevisiae

The constructed plasmids pUC57-ADHI-ILV2-natNT2 and pUC57-natNT2-TPI1-ILV2n were BamHI- and SalI-linearized and used to transform the S. cerevisiae BY4742 parental strain as described above. The transformants were selected on a solid YPD medium supplemented with nourseothricin (100 μg/mL). The selected transformants were stabilized by alternating cultivation in a non-selective media for 12-14 generations followed by shifting to a selective media containing nourseothricin. The expression system for detecting the presence of the desired plasmid construct in the genome of the stable transformants was confirmed by diagnostic PCR.

### EXAMPLES

The following examples are intended to guide those skilled in the art in the practice of this invention. They should not be construed to limit the scope of the invention, which is defined by the claims.

### Example 1—Glycerol Overproduction in Transformed S. Cerevisiae

The selected S. cerevisiae transformants BY4742/ILV2 and BY4742/ILV2n expressing truncated and native form of ILV2 gene, respectively, and the parental strain BY4742 as a control, were each incubated in separate fermentation broths comprising yeast extract 5 g/L, peptone 10 g/L, D-glucose 50 g/L. Fermentation was carried out at a temperature of 30° C. with limited aeration using a rotary shaker at a setting of 120 revolutions/min. An initial biomass concentration of 1 g/L was used for fermentation. On the second day of fermentation, the recombinant strain BY4742/ILV2 possessed approximately 10% growth retardation as compared to parental strain BY4742 (Table 1). The recombinant strain BY4742/ILV2 produced more than a 4-fold increase of glycerol production as compared to the parental strain, reaching 4 g of glycerol/L (Table 1). Ethanol synthesis of the recombinant strain BY4742/ILV2 was 1.8-fold reduced as compared to the parental strain (Table 1). During fermentation, biomass accumulation of strain BY4742/ILV2n was similar to that of parental strain. Strain BY4742/ILV2n produced a slight increase in the amount of glycerol (1.1 g/L) when compared to parental strain BY4742 (0.9 g/L). Ethanol synthesis of the strain was 1.3-fold reduced reaching 13 g/L (Table 1). Glucose consumption and acetate production were approximately on the same level for all analyzed strains. To prove overexpression of both versions of ILV2 gene the specific activity of Ilv2 of the constructed strains was assayed. Specific activities of Ilv2 for strains BY4742/ILV2 and BY4742/ILV2n were increased on 46 and 59%, respectively, as compared to that of parental strain (Table 1).

TABLE 1
Biomass, glycerol, ethanol and acetic acid synthesis, glucose
consumption, specific activity of Ilv2 of S. cerevisiae strains
on the second day of 5% glucose fermentation.

| | | | | | | Ilv2 |
|---|---|---|---|---|---|---|
| | | | | | | specific |
| | | | | | | activity |
| | | Analite (g/L) | | | | (U/mg |
| | Biomass | | Acetic | | | of |
| Strains | (g/L) | Glucose | acid | Ethanol | Glycerol | protein) |
| BY4742 | 2.25 | 0.5 | 0.3 | 17.1 | 0.9 | 74.4 |
| BY4742/ | 2.01 | 0.6 | 0.4 | 9.4 | 4.0 | 108.4 |
| ILV2 | | | | | | |
| BY4742/ | 2.21 | 0.3 | 0.4 | 13.0 | 1.1 | 118.0 |
| ILV2n | | | | | | |

### Further Transformed Yeast Overproducing Glycerol

S. cerevisiae strains may be engineered using in part the ILV2 gene with other genes that are involved the synthesis and degradation of glycerol to construct robust S. cerevisiae strains that are capable of effective glycerol production from glucose under anaerobic conditions. Strains expressing mitochondrial (native) and cytosolic ILV2 may be generated on the background of industrial ethanol producing strain AS400. A cytosolic form of ILV2 may be expressed in strains with altered activity of Pdc (pyruvate decarboxylase), Tpi (triosephosphate isomerase) or Adh (alcohol dehydrogenase) and their combinations.

From the foregoing it will be seen that this invention is one well adapted to attain all ends and objectives herein-above set forth, together with the other advantages which are obvious and which are inherent to the invention.

Since many possible embodiments may be made of the invention without departing from the scope thereof, it is to be understood that all matters herein set forth or shown in the accompanying drawings are to be interpreted as illustrative, and not in a limiting sense.

### REFERENCES

The present application cites several references, summarized herein below. Each such citation is to aid one of ordinary skill in the art in better understanding the present invention and to find sources of sequences, recombinant techniques, tests and other routine information that would enable one of ordinary skill to practice any of numerous embodiments of the present invention. Accordingly, each reference cited is incorporated herein in its entirety by reference, excepting such parts of if such references that contain information that conflicts with the information taught herein, in which case the present disclosure shall be deemed controlling over the incorporated reference.

Wang, Z., Zhuge, J., Fang, H., Prior, B., (2001) Glycerol production by microbial fermentation: A review; Biotechnology Advances 19:201-223.

Drewke, C., Thielen, J., Ciriacy, M. (1990) Ethanol formation in adh0 mutants reveals the existence of a novel acetaldehyde-reducing activity in Saccharomyces cerevisiae; J Bacteriol. 172(7):3909-3917.

Nevoigt E, Stahl U., (1996) Reduced pyruvate decarboxylase and increased glycerol-3-phosphate dehydrogenase [NAD⁺] levels enhance glycerol production in Saccharomyces cerevisiae. Yeast 12:1331-1337.

Pronk, J., Steensma, H, Van Dijken, J. (1996) Pyruvate Metabolism in Saccharomyces cerevisiae; Yeast 12:1607-1633.

Overkamp, K., Bakker, B., Kötter, P., Luttik, M., Van Dijken, J., Pronk, J. (2002) Metabolic engineering of glycerol production in Saccharomyces cerevisiae; Appl Environ Microbiol. 68(6):2814-21.

Giaever G, Chu A M, Ni L, Connelly C, Riles L, Véronneau S, Dow S, Lucau-Danila A, Anderson K, André B, Arkin A P, Astromoff A, El-Bakkoury M, Bangham R, Benito R, Brachat S, Campanaro S, Curtiss M, Davis K, Deutschbauer A, Entian K D, Flaherty P, Foury F, Garfinkel D J, Gerstein M, Gotte D, Güldener U, Hegemann J H, Hempel S, Herman Z, Jaramillo D F, Kelly D E, Kelly S L, Kotter P, LaBonte D, Lamb D C, Lan N, Liang H, Liao H, Liu L, Luo C, Lussier M, Mao R, Menard P, Ooi S L, Revuelta J L, Roberts C J, Rose M, Ross-Macdonald P, Scherens B, Schimmack G, Shafer B, Shoemaker D D, Sookhai-Mahadeo S, Storms R K, Strathern J N, Valle G, Voet M, Volckaert G, Wang C Y, Ward T R, Wilhelmy J, Winzeler E A, Yang Y, Yen G, Youngman E, Yu K, Bussey H, Boeke J D, Snyder M, Philippsen P, Davis R W, Johnston M. (2002) Functional profiling of the Saccharomyces cerevisiae genome; Nature 25;418(6896):387-91.

Sambrook J., Russell D. (2001) Rapid isolation of yeast DNA. In: Sambrook J & Russell D W, eds. Molecular Cloning, a Laboratory Manual, New York: Cold Spring Harbor Laboratory Press, pp. 631-632.

Taxis C, Knop M. (2006) System of centromeric, episomal, and integrative vectors based on drug resistance markers for Saccharomyces cerevisiae; Biotechniques. 40:73-78.

Urano, et al, U.S. Pat. No. 8,232,089, Cytosolic isobutanol pathway localization for the production of isobutanol.

Chen et al (2011) Increased isobutanol production in Saccharomyces cerevisiae by overexpression of genes in valine metabolism; Biotechnology for Biofuels, 4:21.

Brat et al (2012) Cytosolic re-localization and optimization of valine synthesis and catabolism enables increased isobutanol production with the yeast Saccharomyces cerevisiae; Biotechnology for Biofuels, 5:65.

What is claimed is:

1. A recombinant nucleic acid molecule comprising:
a truncated portion of a gene encoding a cytosol located acetolactate synthase activity, wherein said gene is operably linked to a non-native promoter to express said acetolactate synthase activity in the cytosol.

2. The nucleic acid molecule of claim 1, wherein said gene encoding the acetolactate synthase activity encodes a protein at least 75% identical to SEQ ID NO: 2.

3. The nucleic acid molecule of claim 1, wherein said truncated gene does not comprise a mitochondrial targeting signal.

4. The nucleic acid molecule of claim 2, wherein said truncated gene is according to SEQ ID NO: 1.

5. The nucleic acid molecule of claim 1, wherein said promoter is an ADH1 promoter.

6. A vector comprising the nucleic acid molecule of claim 1.

7. The vector of claim 6, further comprising a selective marker.

8. The vector of claim 6, wherein said selective marker is a natNT2 gene.

9. The vector of claim 8 corresponding to SEQ ID NO: 9.

10. A host cell comprising the vector of claim 6.

11. The host cell of claim 10, wherein said host cell is a S. cerevisiae cell.

12. A yeast strain comprising a recombinant nucleic acid molecule comprising a truncated portion of a gene encoding a cytosol located acetolactate synthase activity, wherein said gene is operably linked to a non-native promoter to express said acetolactate synthase activity in the cytosol.

13. The yeast strain of claim 12, wherein said truncated gene does not comprise a mitochondrial targeting signal.

14. The yeast strain of claim 13, wherein said truncated gene is according to SEQ ID NO: 1.

15. The yeast strain of claim 14, wherein said promoter element is an ADH1 promoter.

16. The yeast strain of claim 15, wherein said recombinant nucleic acid molecule is according to SEQ ID NO: 1.

17. The yeast strain of claim 12, wherein said yeast strain is S. cerevisiae.

18. A method for enhancing glycerol production in yeast comprising:
growing the yeast strain of claim 12 anaerobically in a culture medium, under conditions that cause the yeast strain to make glycerol.

19. The method of claim 18, wherein an amount of glycerol produced by said yeast strain is more than four times greater than an amount of glycerol produced by a corresponding yeast strain that does not contain said recombinant nucleic acid molecule but is otherwise identical to said yeast strain.

20. The method of claim 19, wherein said yeast strain is S. cerevisiae.


SEQUENCE LISTING (37 sequences)

SEQ ID NO: 1 - DNA, 2082 bp, Saccharomyces cerevisiae
atggagcctgctccaagtttcaatgttgatccattagaacagcccgctgaaccttcaaaattggctaagaaactacgcgctgagcctgacatggatacctctttcgtcggtttaactggtggtcaaatatttaacgaaatgatgtccagacaaaacgttgatactgtatttggttatccaggtggtgctatcctacctgtttacgatgccattcataacagtgataaattcaacttcgttcttccaaaacacgaacaaggtgccggtcacatggcagaaggctacgccagagcttctggtaaaccaggtgttgtcttggttacttctgggccaggtgccaccaatgtcgttactccaatggcagatgcctttgcagacgggattccaatggttgtctttacagggcaagtcccaactagtgctatcggtactgatgctttccaagaggctgacgtcgttggtatttctagatcttgtacgaaatggaatgtcatggtcaagtccgtggaagaattgccattgcgtattaacgaggcttttgaaattgccacgagcggtagaccgggaccagtcttggtcgatttaccaaaggatgttacagcagctatcttaagaaatccaattccaacaaaaacaactcttccatcaaacgcactaaaccaattaaccagtcgcgcacaagatgaatttgtcatgcaaagtatcaataaagcagcagatttgatcaacttggcaaagaaacctgtcttatacgtcggtgctggtattttaaaccatgcagatggtccaagattactaaaagaattaagtgaccgtgctcaaatacctgtcaccactactttacaaggtttaggttcattcgaccaagaagatccaaaatcattggatatgcttggtatgcacggttgtgctactgccaacctggcagtgcaaaatgccgacttgataattgcagttggtgctagattcgacgaccgtgtcactggtaatatttctaaattcgctccagaagctcgtcgtgcagctgccgagggtagaggtggtattattcatttcgaggttagtccaaaaaacataaacaaggttgttcaaactcaaatagcagtggaaggtgatgctacgaccaatctgggcaaaatgatgtcaaagattttcccagttaaggagaggtctgaatggtttgctcaaataaataaatggaagaaggaatacccatacgcttatatggaggagactccaggatctaaaattaaaccacagacggttataaagaaactatccaaggttgccaacgacacaggaagacatgtcattgttacaacgggtgtggggcaacatcaaatgtgggctgctcaacactggacatggagaaatccacatactttcatcacatcaggtggtttaggtacgatgggttacggtctccctgccgccatcggtgctcaagttgcaaagccagaatctttggttattgacattgatggtgacgcatcctttaacatgactctaacggaattgagttctgccgttcaagctggtactccagtgaagattttgattttgaacaatgaagagcaaggtatggttactcaatggcaatccctgttctacgaacatcgttattcccacacacatcaattgaaccctgatttcataaaactagcggaggctatgggtttaaaaggtttaagagtcaagaagcaagaggaattggacgctaagttgaaagaattcgtttctaccaagggcccagttttgcttgaagtggaagttgataaaaaagttcctgttttgccaatggtggcaggtggtagcggtctagacgagttcataaattttgacccagaagttgaaagacaacagactgaattacgtcataagcgtacaggcggtaagcactgaatttcaaaaacatttatttcaaaagcattttcagtaaaaaatgcagactttattattatttaatcgtgcttcttatatatgacattctaccaaatcggtagtcatgtatatttttttcgtatatactttatatatttttttctaaaaaactaatgacggctaaaattaagtcatagatga

SEQ ID NO: 2 - protein, 633 aa, Saccharomyces cerevisiae
MEPAPSFNVDPLEQPAEPSKLAKKLRAEPDMDTSFVGLTGGQIFNEMMSRQNVDTVFGYPGGAILPVYDAIHNSDKFNFVLPKHEQGAGHMAEGYARASGKPGVVLVTSGPGATNVVTPMADAFADGIPMVVFTGQVPTSAIGTDAFQEADVVGISRSCTKWNVMVKSVEELPLRINEAFEIATSGRPGPVLVDLPKDVTAAILRNPIPTKTTLPSNALNQLTSRAQDEFVMQSINKAADLINLAKKPVLYVGAGILNHADGPRLLKELSDRAQIPVTTTLQGLGSFDQEDPKSLDMLGMHGCATANLAVQNADLIIAVGARFDDRVTGNISKFAPEARRAAAEGRGGIIHFEVSPKNINKVVQTQIAVEGDATTNLGKMMSKIFPVKERSEWFAQINKWKKEYPYAYMEETPGSKIKPQTVIKKLSKVANDTGRHVIVTTGVGQHQMWAAQHWTWRNPHTFITSGGLGTMGYGLPAAIGAQVAKPESLVIDIDGDASFNMTLTELSSAVQAGTPVKILILNNEEQGMVTQWQSLFYEHRYSHTHQLNPDFIKLAEAMGLKGLRVKKQEELDAKLKEFVSTKGPVLLEVEVDKKVPVLPMVAGGSGLDEFINFDPEVERQQTELRHKRTGGKH

SEQ ID NO: 3 - DNA, 2633 bp, Saccharomyces cerevisiae
atgatcagacaatctacgctaaaaaacttcgctattaagcgttgctttcaacatatagcataccgcaacacacctgccatgagatcagtagctctcgcgcagcgcttttatagttcgtcttcccgttattacagtgcgtctccattaccagcctctaaaaggccagagcctgctccaagtttcaatgttgatccattagaacagcccgctgaaccttcaaaattggctaagaaactacgcgctgagcctgacatggatacctctttcgtcggtttaactggtggtcaaatatttaacgaaatgatgtccagacaaaacgttgatactgtatttggttatccaggtggtgctatcctacctgtttacgatgccattcataacagtgataaattcaacttcgttcttccaaaacacgaacaaggtgccggtcacatggcagaaggctacgccagagcttctggtaaaccaggtgttgtcttggttacttctgggccaggtgccaccaatgtcgttactccaatggcagatgcctttgcagacgggattccaatggttgtctttacagggcaagtcccaactagtgctatcggtactgatgctttccaagaggctgacgtcgttggtatttctagatcttgtacgaaatggaatgtcatggtcaagtccgtggaagaattgccattgcgtattaacgaggcttttgaaattgccacgagcggtagaccgggaccagtcttggtcgatttaccaaaggatgttacagcagctatcttaagaaatccaattccaacaaaaacaactcttccatcaaacgcactaaaccaattaaccagtcgcgcacaagatgaatttgtcatgcaaagtatcaataaagcagcagatttgatcaacttggcaaagaaacctgtcttatacgtcggtgctggtattttaaaccatgcagatggtccaagattactaaaagaattaagtgaccgtgctcaaatacctgtcaccactactttacaaggtttaggttcattcgaccaagaagatccaaaatcattggatatgcttggtatgcacggttgtgctactgccaacctggcagtgcaaaatgccgacttgataattgcagttggtgctagattcgacgaccgtgtcactggtaatatttctaaattcgctccagaagctcgtcgtgcagctgccgagggtagaggtggtattattcatttcgaggttagtccaaaaaacataaacaaggttgttcaaactcaaatagcagtggaaggtgatgctacgaccaatctgggcaaaatgatgtcaaagattttcccagttaaggagaggtctgaatggtttgctcaaataaataaatggaagaaggaatacccatacgcttatatggaggagactccaggatctaaaattaaaccacagacggttataaagaaactatccaaggttgccaacgacacaggaagacatgtcattgttacaacgggtgtggggcaacatcaaatgtgggctgctcaacactggacatggagaaatccacatactttcatcacatcaggtggtttaggtacgatgggttacggtctccctgccgccatcggtgctcaagttgcaaagccagaatctttggttattgacattgatggtgacgcatcctttaacatgactctaacggaattgagttctgccgttcaagctggtactccagtgaagattttgattttgaacaatgaagagcaaggtatggttactcaatggcaatccctgttctacgaacatcgttattcccacacacatcaattgaaccctgatttcataaaactagcggaggctatgggtttaaaaggtttaagagtcaagaagcaagaggaattggacgctaagttgaaagaattcgtttctaccaagggcccagttttgcttgaagtggaagttgataaaaaagttcctgttttgccaatggtggcaggtggtagcggtctagacgagttcataaattttgacccagaagttgaaagacaacagactgaattacgtcataagcgtacaggcggtaagcactgaatttcaaaaacatttatttcaaaagcattttcagtaaaaaatgcagactttattattatttaatcgtgcttcttatatatgacattctaccaaatcggtagtcatgtatatttttttcgtatatactttatatatttttttctaaaaaactaatgacggctaaaattaagtcatagatgaataataagttcaattcaagtgagttggtagtatttgataaatctaaagtggatacgtagcatatgtattcaaatggtgtgttttaactcgtcggacacgacttctttctcgttttgtcaatcatcaaaaatatttctcgaaaaggggccgtcaaaaattgcaagcttggaaaatacaaactggactatgtatttaaaggttatagtttataataaagcataagaacagtggccaatacgaatttaaccgctttatagaaatggctatcttaaaaagaggagctagaaaaaaggtacatcaggagccagctaaacgctctgcgaatatcaagaaagctacttttgattcctcgaagaagaaagaagttggtgtgtctgatctgacgcttc

SEQ ID NO: 4 - protein, 661 aa, Saccharomyces cerevisiae
MRSVALAQRFYSSSSRYYSASPLPASKRPEPAPSFNVDPLEQPAEPSKLAKKLRAEPDMDTSFVGLTGGQIFNEMMSRQNVDTVFGYPGGAILPVYDAIHNSDKFNFVLPKHEQGAGHMAEGYARASGKPGVVLVTSGPGATNVVTPMADAFADGIPMVVFTGQVPTSAIGTDAFQEADVVGISRSCTKWNVMVKSVEELPLRINEAFEIATSGRPGPVLVDLPKDVTAAILRNPIPTKTTLPSNALNQLTSRAQDEFVMQSINKAADLINLAKKPVLYVGAGILNHADGPRLLKELSDRAQIPVTTTLQGLGSFDQEDPKSLDMLGMHGCATANLAVQNADLIIAVGARFDDRVTGNISKFAPEARRAAAEGRGGIIHFEVSPKNINKVVQTQIAVEGDATTNLGKMMSKIFPVKERSEWFAQINKWKKEYPYAYMEETPGSKIKPQTVIKKLSKVANDTGRHVIVTTGVGQHQMWAAQHWTWRNPHTFITSGGLGTMGYGLPAAIGAQVAKPESLVIDIDGDASFNMTLTELSSAVQAGTPVKILILNNEEQGMVTQWQSLFYEHRYSHTHQLNPDFIKLAEAMGLKGLRVKKQEELDAKLKEFVSTKGPVLLEVEVDKKVPVLPMVAGGSGLDEFINFDPEVERQQTELRHKRTGGKH

SEQ ID NO: 5 - DNA, 691 bp, Saccharomyces cerevisiae
atatggacttcctcttttctggcaaccaaacccatacatcgggattcctataataccttcgttggtctccctaacatgtaggtggcggaggggagatatacaatagaacagataccagacaagacataatgggctaaacaagactacaccaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaaagtttgctgtcttgctatcaagtataaatagacctgcaattattaatcttttgtttcctcgtcattgttctcgttccctttcttccttgtttctttttctgcacaatatttcaagctataccaagcatacaatcaactatctcatataca

SEQ ID NO: 6 - DNA, 605 bp, Saccharomyces cerevisiae
tgagggagacctaactacatagtgtttaaagattacggatatttaacttacttagaataatgccatttttttgagttataataatcctacgttagtgtgagcgggatttaaactgtgaggaccttaatacattcagacacttctgcggtatcaccctacttattcccttcgagattatatctaggaacccatcaggttggtggaagattacccgttctaagacttttcagcttcctctattgatgttacacctggacaccccttttctggcatccagtttttaatcttcagtggcatgtgagattctccgaaattaattaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaa

SEQ ID NO: 7 - DNA, 1391 bp, Streptomyces noursei
gtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaccactcttgacgacacggcttaccggtaccgcaccagtgtcccgggggacgccgaggccatcgaggcactggatgggtccttcaccaccgacaccgtcttccgcgtcaccgccaccggggacggcttcaccctgcgggaggtgccggtggacccgcccctgaccaaggtgttccccgacgacgaatcggacgacgaatcggacgacggggaggacggcgacccggactcccggacgttcgtcgcgtacggggacgacggcgacctggcgggcttcgtggtcatctcgtactcggcgtggaaccgccggctgaccgtcgaggacatcgaggtcgccccggagcaccgggggcacggggtcgggcgcgcgttgatggggctcgcgacggagttcgccggcgagcggggcgccgggcacctctggctggaggtcaccaacgtcaacgcaccggcgatccacgcgtaccggcggatggggttcaccctctgcggcctggacaccgccctgtacgacggcaccgcctcggacggcgagcggcaggcgctctacatgagcatgccctgcccctaatctcgaggcgaatttcttatgatttatgatttttattattaaataagttataaaaaaaataagtgtatacaaattttaaagtgactcttaggttttaaaacgaaaattcttgttcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcag

SEQ ID NO: 8 - DNA, 34 bp, Artificial Sequence
  LoxP- genetic tool for marker rescue
ataacttcgtatagcatacattatacgaagttat

SEQ ID NO: 9 - DNA, 6822 bp, Artificial Sequence
  pUC57-ADH1-ILV2(truncated)-natNT2 plasmid sequence
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatatcggatccatatggacttcctcttttctggcaaccaaacccatacatcgggattcctataataccttcgttggtctccctaacatgtaggtggcggaggggagatatacaatagaacagataccagacaagacataatgggctaaacaagactacaccaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaaagtttgctgtcttgctatcaagtataaatagacctgcaattattaatcttttgtttcctcgtcattgttctcgttccctttcttccttgtttctttttctgcacaatatttcaagctataccaagcatacaatcaactatctcatatacagtcgacatggagcctgctccaagtttcaatgttgatccattagaacagcccgctgaaccttcaaaattggctaagaaactacgcgctgagcctgacatggatacctctttcgtcggtttaactggtggtcaaatatttaacgaaatgatgtccagacaaaacgttgatactgtatttggttatccaggtggtgctatcctacctgtttacgatgccattcataacagtgataaattcaacttcgttcttccaaaacacgaacaaggtgccggtcacatggcagaaggctacgccagagcttctggtaaaccaggtgttgtcttggttacttctgggccaggtgccaccaatgtcgttactccaatggcagatgcctttgcagacgggattccaatggttgtctttacagggcaagtcccaactagtgctatcggtactgatgctttccaagaggctgacgtcgttggtatttctagatcttgtacgaaatggaatgtcatggtcaagtccgtggaagaattgccattgcgtattaacgaggcttttgaaattgccacgagcggtagaccgggaccagtcttggtcgatttaccaaaggatgttacagcagctatcttaagaaatccaattccaacaaaaacaactcttccatcaaacgcactaaaccaattaaccagtcgcgcacaagatgaatttgtcatgcaaagtatcaataaagcagcagatttgatcaacttggcaaagaaacctgtcttatacgtcggtgctggtattttaaaccatgcagatggtccaagattactaaaagaattaagtgaccgtgctcaaatacctgtcaccactactttacaaggtttaggttcattcgaccaagaagatccaaaatcattggatatgcttggtatgcacggttgtgctactgccaacctggcagtgcaaaatgccgacttgataattgcagttggtgctagattcgacgaccgtgtcactggtaatatttctaaattcgctccagaagctcgtcgtgcagctgccgagggtagaggtggtattattcatttcgaggttagtccaaaaaacataaacaaggttgttcaaactcaaatagcagtggaaggtgatgctacgaccaatctgggcaaaatgatgtcaaagattttcccagttaaggagaggtctgaatggtttgctcaaataaataaatggaagaaggaatacccatacgcttatatggaggagactccaggatctaaaattaaaccacagacggttataaagaaactatccaaggttgccaacgacacaggaagacatgtcattgttacaacgggtgtggggcaacatcaaatgtgggctgctcaacactggacatggagaaatccacatactttcatcacatcaggtggtttaggtacgatgggttacggtctccctgccgccatcggtgctcaagttgcaaagccagaatctttggttattgacattgatggtgacgcatcctttaacatgactctaacggaattgagttctgccgttcaagctggtactccagtgaagattttgattttgaacaatgaagagcaaggtatggttactcaatggcaatccctgttctacgaacatcgttattcccacacacatcaattgaaccctgatttcataaaactagcggaggctatgggtttaaaaggtttaagagtcaagaagcaagaggaattggacgctaagttgaaagaattcgtttctaccaagggcccagttttgcttgaagtggaagttgataaaaaagttcctgttttgccaatggtggcaggtggtagcggtctagacgagttcataaattttgacccagaagttgaaagacaacagactgaattacgtcataagcgtacaggcggtaagcactgaatttcaaaaacatttatttcaaaagcattttcagtaaaaaatgcagactttattattatttaatcgtgcttcttatatatgacattctaccaaatcggtagtcatgtatatttttttcgtatatactttatatatttttttctaaaaaactaatgacggctaaaattaagtcatagatgactgcagaggcctgcatgcaagcttggcgcgccagatctataacttcgtatagcatacattatacgaagttatcttaactatgcggcatcagagcagattgtactgagagtgcagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaccactcttgacgacacggcttaccggtaccgcaccagtgtcccgggggacgccgaggccatcgaggcactggatgggtccttcaccaccgacaccgtcttccgcgtcaccgccaccggggacggcttcaccctgcgggaggtgccggtggacccgcccctgaccaaggtgttccccgacgacgaatcggacgacgaatcggacgacggggaggacggcgacccggactcccggacgttcgtcgcgtacggggacgacggcgacctggcgggcttcgtggtcatctcgtactcggcgtggaaccgccggctgaccgtcgaggacatcgaggtcgccccggagcaccgggggcacggggtcgggcgcgcgttgatggggctcgcgacggagttcgccggcgagcggggcgccgggcacctctggctggaggtcaccaacgtcaacgcaccggcgatccacgcgtaccggcggatggggttcaccctctgcggcctggacaccgccctgtacgacggcaccgcctcggacggcgagcggcaggcgctctacatgagcatgccctgcccctaatctcgaggcgaatttcttatgatttatgatttttattattaaataagttataaaaaaaataagtgtatacaaattttaaagtgactcttaggttttaaaacgaaaattcttgttcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggataacttcgtatagcatacattatacgaagttatagatctggcgcgccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 10 - DNA, 7356 bp, Artificial Sequence
  pUC57-natNT2-TPI1-ILV2n plasmid sequence
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatatcggatcctctagagtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaccactcttgacgacacggcttaccggtaccgcaccagtgtcccgggggacgccgaggccatcgaggcactggatgggtccttcaccaccgacaccgtcttccgcgtcaccgccaccggggacggcttcaccctgcgggaggtgccggtggacccgcccctgaccaaggtgttccccgacgacgaatcggacgacgaatcggacgacggggaggacggcgacccggactcccggacgttcgtcgcgtacggggacgacggcgacctggcgggcttcgtggtcatctcgtactcggcgtggaaccgccggctgaccgtcgaggacatcgaggtcgccccggagcaccgggggcacggggtcgggcgcgcgttgatggggctcgcgacggagttcgccggcgagcggggcgccgggcacctctggctggaggtcaccaacgtcaacgcaccggcgatccacgcgtaccggcggatggggttcaccctctgcggcctggacaccgccctgtacgacggcaccgcctcggacggcgagcggcaggcgctctacatgagcatgccctgcccctaatctcgaggcgaatttcttatgatttatgatttttattattaaataagttataaaaaaaataagtgtatacaaattttaaagtgactcttaggttttaaaacgaaaattcttgttcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagtctagaggatcctgagggagacctaactacatagtgtttaaagattacggatatttaacttacttagaataatgccatttttttgagttataataatcctacgttagtgtgagcgggatttaaactgtgaggaccttaatacattcagacacttctgcggtatcaccctacttattcccttcgagattatatctaggaacccatcaggttggtggaagattacccgttctaagacttttcagcttcctctattgatgttacacctggacaccccttttctggcatccagtttttaatcttcagtggcatgtgagattctccgaaattaattaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaaggtaccatgatcagacaatctacgctaaaaaacttcgctattaagcgttgctttcaacatatagcataccgcaacacacctgccatgagatcagtagctctcgcgcagcgcttttatagttcgtcttcccgttattacagtgcgtctccattaccagcctctaaaaggccagagcctgctccaagtttcaatgttgatccattagaacagcccgctgaaccttcaaaattggctaagaaactacgcgctgagcctgacatggatacctctttcgtcggtttaactggtggtcaaatatttaacgaaatgatgtccagacaaaacgttgatactgtatttggttatccaggtggtgctatcctacctgtttacgatgccattcataacagtgataaattcaacttcgttcttccaaaacacgaacaaggtgccggtcacatggcagaaggctacgccagagcttctggtaaaccaggtgttgtcttggttacttctgggccaggtgccaccaatgtcgttactccaatggcagatgcctttgcagacgggattccaatggttgtctttacagggcaagtcccaactagtgctatcggtactgatgctttccaagaggctgacgtcgttggtatttctagatcttgtacgaaatggaatgtcatggtcaagtccgtggaagaattgccattgcgtattaacgaggcttttgaaattgccacgagcggtagaccgggaccagtcttggtcgatttaccaaaggatgttacagcagctatcttaagaaatccaattccaacaaaaacaactcttccatcaaacgcactaaaccaattaaccagtcgcgcacaagatgaatttgtcatgcaaagtatcaataaagcagcagatttgatcaacttggcaaagaaacctgtcttatacgtcggtgctggtattttaaaccatgcagatggtccaagattactaaaagaattaagtgaccgtgctcaaatacctgtcaccactactttacaaggtttaggttcattcgaccaagaagatccaaaatcattggatatgcttggtatgcacggttgtgctactgccaacctggcagtgcaaaatgccgacttgataattgcagttggtgctagattcgacgaccgtgtcactggtaatatttctaaattcgctccagaagctcgtcgtgcagctgccgagggtagaggtggtattattcatttcgaggttagtccaaaaaacataaacaaggttgttcaaactcaaatagcagtggaaggtgatgctacgaccaatctgggcaaaatgatgtcaaagattttcccagttaaggagaggtctgaatggtttgctcaaataaataaatggaagaaggaatacccatacgcttatatggaggagactccaggatctaaaattaaaccacagacggttataaagaaactatccaaggttgccaacgacacaggaagacatgtcattgttacaacgggtgtggggcaacatcaaatgtgggctgctcaacactggacatggagaaatccacatactttcatcacatcaggtggtttaggtacgatgggttacggtctccctgccgccatcggtgctcaagttgcaaagccagaatctttggttattgacattgatggtgacgcatcctttaacatgactctaacggaattgagttctgccgttcaagctggtactccagtgaagattttgattttgaacaatgaagagcaaggtatggttactcaatggcaatccctgttctacgaacatcgttattcccacacacatcaattgaaccctgatttcataaaactagcggaggctatgggtttaaaaggtttaagagtcaagaagcaagaggaattggacgctaagttgaaagaattcgtttctaccaagggcccagttttgcttgaagtggaagttgataaaaaagttcctgttttgccaatggtggcaggtggtagcggtctagacgagttcataaattttgacccagaagttgaaagacaacagactgaattacgtcataagcgtacaggcggtaagcactgaatttcaaaaacatttatttcaaaagcattttcagtaaaaaatgcagactttattattatttaatcgtgcttcttatatatgacattctaccaaatcggtagtcatgtatatttttttcgtatatactttatatatttttttctaaaaaactaatgacggctaaaattaagtcatagatgaataataagttcaattcaagtgagttggtagtatttgataaatctaaagtggatacgtagcatatgtattcaaatggtgtgttttaactcgtcggacacgacttctttctcgttttgtcaatcatcaaaaatatttctcgaaaaggggccgtcaaaaattgcaagcttggaaaatacaaactggactatgtatttaaaggttatagtttataataaagcataagaacagtggccaatacgaatttaaccgctttatagaaatggctatcttaaaaagaggagctagaaaaaaggtacatcaggagccagctaaacgctctgcgaatatcaagaaagctacttttgattcctcgaagaagaaagaagttggtgtgtctgatctgacgcttcgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 11 - DNA, 50 bp, Artificial Sequence
  Ko574 PCR primer
caatcaactatctcatatacagtcgacatggagcctgctccaagtttcaa

SEQ ID NO: 12 - DNA, 32 bp, Artificial Sequence
  Ko575 PCR primer
aaactgcagtcatctatgacttaattttagcc

SEQ ID NO: 13 - DNA, 30 bp, Artificial Sequence
  Ko572 PCR primer
cgcggatccatatggacttcctcttttctg

SEQ ID NO: 14 - DNA, 50 bp, Artificial Sequence
  Ko573 PCR Primer
ttgaaacttggagcaggctccatgtcgactgtatatgagatagttgattg

SEQ ID NO: 15 - DNA, 78 bp, Artificial Sequence
  OK19 PCR primer
cccaagcttggcgcgccagatctataacttcgtatagcatacattatacgaagttatcttaactatgcggcatcagag

SEQ ID NO: 16 - DNA, 76 bp, Artificial Sequence
  OK20 PCR primer
aagcttggcgcgccagatctataacttcgtataatgtatgctatacgaagttatccgagattcatcaactcattgc

SEQ ID NO: 17 - DNA, 30 bp, Artificial Sequence
  LY7 PCR primer
cgggatcctgagggagacctaactacatag

SEQ ID NO: 18 - DNA, 51 bp, Artificial Sequence
  LY8 PCR primer
tagcgtagattgtctgatcatggtaccttttagtttatgtatgtgtttttt

SEQ ID NO: 19 - DNA, 51 bp, Artificial Sequence
  LY9 PCR primer
aaaaaacacatacataaactaaaaggtaccatgatcagacaatctacgcta

SEQ ID NO: 20 - DNA, 28 bp, Artificial Sequence
  LY10 PCR primer
gcgtcgacgaagcgtcagatcagacaca

SEQ ID NO: 21 - DNA, 37 bp, Artificial Sequence
  Ko446 PCR primer
ccgggatcctctagagtgatgacggtgaaaacctctg

SEQ ID NO: 22 - DNA, 38 bp, Artificial Sequence
  Ko448 PCR Primer
ccgggatcctctagactgaggacataaaatacacaccg

SEQ ID NO: 23 - protein, 687 aa, Saccharomyces kudriavzevil
MIRQSTLKNFALKRCFQQIAYRSTPAMRSVALAQRFYSSSSRYYSASPLPVSKRPEPAPSFNVEPLEQAPEPSKLAKRLRTEPDMDTSFVGLTGGQIFNEMMSRQNVDTVFGYPGGAILPVYDAIHNSDKFNFVLPKHEQGAGHMAEGYARASGKPGVVLVTSGPGATNVVTPMADAFADGIPMVVFTGQVPTSAIGTDAFQEADVVGISRSCTKWNVMVKTVEELPLRINEAFEIATSGRPGPVLVDLPKDVTAAILRNPIPTKTTLPSNALNQLTSHAQDEFVMQSISKAADLINLAKKPVLYVGAGILNNADGPRLLKELSERAQIPVTTTLQGLGSFDQEDPKSLDMLGMHGCVTANLAVQNADLLIAVGARFDDRVTCNIAKFAPEARRAAAEGRGGIVHFEVVPKNINKVVETQIAVEGDATSNLDKMMPKIFPVKERSEWFGQINKWKKEFPYAYMMETPGSKIKPQTVITKLSKIANDTGRHVIVTTGVGQHQMWAAQHWTWKNPRTFITSGGLGTMGYGLPSAIGAQVAKPESLVIDIDGDASFNMTLTELSSAVQAGTPVKILILNNEEQGMVTQWQSLFYEHRYSHTHQLNPDFIKLAEAMGLKGLRVKKQEELDAKLKEFVSTKGPVLLEVEVDKKVPVLPMVPAGKGLDESINFDPEVEKQQTELRHKRTGGKY

SEQ ID NO: 24 - protein, 684 aa, Nauclea orientalis
MLRQAALKNVTAKRCFQQFAMKSSTVSVATRYYRSTPRFYSSASQATSVRPEPAPSFNVDPAENATSKPSKLAKKIRQEPQMDTSFVGLSGGQIFNEMMKRQNVDTVFGYPGGAILPVYDAIYNSEAFNFVLPKHEQGAGHMAEGYARASGKPGVVLVTSGPGATNVVTPMADALADGIPMVVFTGQVPTSAIGTDAFQEADVVGISRSCTKWNVMVKSVAELPLRINEAFEIAMSGRPGPVLVDLPKDVTAAILREPIPIKSTLPSGTLTQIKSIAQEDFLADSINRAADLINLAKKPVLYVGNGIFNNEDGPRLLKELSERAQIPVTTTIQGLGAFDQEDPKSLDMLGMHGCATANLAMQNADLIIAVGARFDDRVTGNIAKFAPEARRAANEGRGGIIHFEISPKNINKVVEAQVAVEGDAAANIDKMLPAIFPIKERTEWFKEINQWKKDYPYDYMKEVPGGKLKPQTVISKLSKVANATGRPVIVTTGVGQHQMWTAQHWTWKHPRSFITSGGLGTMGYGLPSAIGAQVAHPEALVIDIDGDASFNMTLTELSSAVQAGTPVKIMILNNEEQGMVTQWQSLFYENRYSHTHQLNPDFMKLADAMGLKGIRVKKQEELDGALKEFVSTPGPVLLEVEVEKKVPVLPMVPAGKGLDEFMSYDPKVEKEQNELRHKRTGGKH

SEQ ID NO: 25 - protein, 685 aa, Naumovozyma dairenensis
MLRQAALKNVATKRGFQQLTLRSSKAPSSIAFRYYKSIPRNYSSVSNAAAVRPEPAPSFNVDPASQPSQKQPVKLSKKLRTEPEMDSSFVGLSGGQIFNEMMKRQNVDTVFGYPGGAILPVYDAIYNSDAFKFVLPKHEQGAGHMAEGYARASGKTGVVLVTSGPGATNVVTPMADALADGIPMVVFTGQVPTSAIGTDAFQEADVVGISRSCTKWNVMVRSVAELPLRINEAFEIANSGRPGPVLVDLPKDVTAAILRNPIPIKSTLPITSQLKSVAEEAYTMESINRAADLINLAKRPVLYVGNGIMNNPDGPRLLKELSDRAQIPVTTTIQGLGAFDQEDPKSLDMLGMHGCATANLAMQNADLVIALGARFDDRVTGNIAKFAPEARRAALEERGGIIHFEISPKNINKVVEAQVAVEGDAASNVSKLLPLVFPVKERKDWFKQINQWKKEYPYSYMKETAESKIKPQTVISKLSKIANGTGKPVVVTTGVGQHQMWAAQHWTWKHPRSFITSGGLGTMGYGLPAAIGAQVARPDAIVIDIDGDASFNMTLTELSSAVQAGVPIKILLLNNEEQGMVTQWQSLFYEHRYSHTHQLNPDFMKLADAMGLKGIRVKNQGELDGALKEFVDYQGPVLLEVEVEKKVPVLPMVPAGKGLDEFINFDPEMEKEQNELRHKRTGGKH

SEQ ID NO: 26 - protein, 677 aa, Candida glabrata
MTRRGLTDPMIRISGVVARACRRSAAYQVCKYSVRAVPRPEPSPSFNVDPDTAAANTGSGVKLSSKLKNVATQKMDNSFVGLTGGQIFNEMMARNNVDTVFGYPGGAILPVYDAIHNSDKFKFVLPKHEQGAGHMAEGYARASGKPGVVLVTSGPGATNVVTPMADALADGIPMVVFTGQVPTTAIGTDAFQEADIVGISRSCTKWNVMVKNVAELPLRINEAFQIATSGRPGPVLVDLPKDVTAAILRTPIPVKSTLPSTALQQLTAGVQDQFVIENIKKSADLINIAKNPVLYVGGGILNNVDGPRLVKELSERAQIPVTTTLQGLGAFDQEDPKSLDMLGMHGCATANLAVQNSDLIIAVGARFDDRVTGNITKFAPEARKAALEGRGGIIHFEITPKNINKVVEAQVAVEGDAASNLSKLIPLVFPVKERPEWFEKINKWKKEFPYSYSLETPGSRIKPQTAIAKLSKIANATSKEVIVTTGVGQHQMWAAQHWTWKNPRTFITSGGLGTMGYGLPSAIGAQVAKPDALVIDIDGDASFNMSLQELSSAVQANAPVKILVLNNEEQGMVTQWQSLFYEHRYSHTHQLNPDFVKLAEAMGMKGMRVKDQAELEKTLKEFVDYQGPVLLEVEVEKKVPVLPMVPAGKGLHEFINYDPEMEREQNELRHKRTGGKH

SEQ ID NO: 27 - protein, 682 aa, Torulaspora delbrueckii
MIRQATIKQFAAKRCFREIASRSAVYGTVRYYGSASRNYSATVASAVASGRPEPAPSFNVDPAAGKPSKLNQKLRTDPQMDSSFIGLTGGQIFHEMMIRHNVDTVFGYPGGAILPVYDAIHDSKSFNFVLPRHEQGAGHMAEGYARASGKPGVVLVTSGPGATNVVTPMADALADGVPMVVFTGQVPTSAIGTDAFQEADVVGISRSCTKWNVMVRSIEELPLRINEAFEIAVSGRPGPVLVDLPKDVTAAILKNPIPTKTTLPSNSLAQLTKRAMDESTNENIVRTADLLNKAKTPVLYVGAGILNNPAGPKLLKELSERAQIPVTTTIQALGAFDQQDPKSLDMLGMHGCATANMAIQNADLILAVGSRFDDRVTLNISKFAPEARRAALENRGGIVHFEICPKNISKVVETQVAVEGDAASNIEKLLPLVVPVKERTEWFAQIEEWKEKYPYAYQKETPGSKIKPQTVISKLSDLANKSDKEVIVTTGVGQHQMWAAQYWTWKSPRSFITSGGLGTMGYGLPAAIGAQVAKPDALVIDIDGDASFNMTLMELSSAVQAKTPVKILLLNNEEQGMVTQWQSLFYEHRYSHTHQLNPDFLKLADAMGLKGLRLSKQEDVDATLKEFIETEGPVLLEVKVEPKVPVLPMVPAGKGLDEFIVFDPKVEMEQNALRHKRTGGKH

SEQ ID NO: 28 - protein, 682 aa, Kazachstania africana
MMLRCPVLRSHALKRCARSKSPFLALRYYTNATKTLNSASAQAVATNRRPEPAPSFDVDPLSSEIRESFGKKAKVDPMKMDNSLVGLTGGQIFNEMMARHNVDTVFGYPGGAILPVYDAIYQSKNFKFVLPKHEQGAGHMAEGYARASGKPGIVLVTSGPGATNVVTPMADALADGIPMVVFTGQVPTTAIGTDAFQEADVVGISRSCTKWNVMIKNVAELPVRINEAFEIATSGRPGPVLVDLPKDVTAAILRDAIPVGSSLPSNALSQLTNKAQDEFVLDNINKAADLINLAKNPVLYVGGGILGNEDGPKLLKQLSERAQIPVTTSLLGLGAFDQEDPKSLDMLGMHGCATANLAMQNADLIIAIGTRFDDRVTGNIAKFAPEARRAALENRGGIIHFEISPKNINKVVEAQIAVEGDATANLQSILPKIFPVKERTEWFAKIKEWKTKYPYSYMKETPNSKLKPQTVISKLSKIANATGKEVIVTTGVGQHQMWAAQHWTWKHPRTFITSGGLGTMGYGLPAAIGAQVAKPDALVIDIDGDASFNMTLTELSSAVQAGAPVKILLLNNEEQGMVTQWQSLFYENRYSHTHQLNPDFIKLAEAMGLKAIKVSKQNQLDKALQEFVEYDGPVLLEVEVEKKVPVLPMVPAGKGLHEFMSFDENTEKEQNELRRNRTNGKY

SEQ ID NO: 29 - protein, 693 aa, Kazachstania naganishii
MLRSQLVKNKALHRATARGYSQNVWTRFVRSAPATCRYTAVRCASRSSSARPEPSPSFDLDPATSLYKASNESSIKRSKLSDKLRSATELDNSLVGLTGGQIFNEMMSRHNVDTVFGYPGGAILPVYDAIYGSENFKFVLPKHEQGAGHMAEGYARASGKPGVVLVTSGPGATNVVTPMADALADGVPMVVFTGQVPTNAIGTDAFQEADVVGISRSCTKWNVMVKNVAELPKRINEAFEIAMSGRPGPVLVDLPKDVTAAVLREAIPTESTLPSLNGGATTGATVFDEFLAPAINKAADLINLAKKPVLYVGNGILNNEDGPRLLKELSDRAQIPVTTTLQGLGAFDQEDPKSLDMLGMHGCATANLAIQNADLIIAVGARFDDRVTGNITKFAPEAKIAAQENRGGIIHFEVSPKNINKVVEAQVAVEGDATENIATMLPKVFPVKHRAEWFKQIEQWKQKFPYAYMKETPGSKIMPQTVIAKLSKLANASGRDVVVTTGVGQHQMWAAQHWTWKKPRTFVTSGGLGTMGFGLPAAIGAQVAKPDAMVIDIDGDASFNMTLTELSSAVQAGTPVKILLLNNEEQGMVTQWQSLFYENRYSHTHQRNPDFQKLCEAMGVKPLKASRQEELDSALEQFVEAEGPVLLEVIVEKKVPVLPMVPAGKGLDEFINFDPEEEKKQNELRHKRTKGKH

SEQ ID NO: 30 - protein, 671 aa, Vanderwaltozyma polyspora
MLRTTSAPVVARRSLQNVLKRSAPLSLTRPLSYCRVRLSQAQERPEPAPSFNVGPTQQTVFPKKDQLKMDKSLIGLTGGEIFHEMMIRNNVDTVFGYPGGAILPVYDAIYNSDKFKFVLPRHEQGAGHMAEGYSRASGKPGVVLVTSGPGATNVVTPMADALADGIPMVVFTGQVPTSAIGTDAFQEADVIGISRSCTKWNVMVKSVEELPRRINEAFEIATSGRPGPVLVDLPKDVTAAILRNPIPVNSTLPSNALNQLVKNAHNEFTEESLSKAASLINIAKRPVLYVGGGILNHEEGPRLLRELSERAQIPVTTSLLGLGAFDQEDPKSLDMLGMHGHAPANLAIQNADLIIAVGARFDDRVTGNIAKFAPEARRAALENRGGIIHFEITAKNINKVVDTQVAVEGDAAANLEKLLPNIFAVKERKDWFDLIEDWKQKYPYSYMKEEPGSRIKPQTIIAKLSKLANESNRETIVTTGVGQHQMWAAQHWTWKHPRTFITSGGLGTMGFGLPSAIGAQVAKPDSMVIDIDGDASFNMTLMEMTSAVQASAPVKVLLLNNEEQGMVTQWQSLFYKHRYSHTHQLNPDFVKLAEAMGFKALRLNKQEDVDAKLKEFVDCEGPVLLEVEVEKKVPVLPMVPAGKGLDEFIKFDPETEREQKELRHKRTQGKH

SEQ ID NO: 31 - protein, 700 aa, Zygosaccharomyces rouxii
MIRQARQMGATRCAVRYAVRNISSNSARSSRNLAYINTVRYHSTTRKQQYSAATATGESTATSNATARRPAPAPSFNVDLTGTPDKLHKKLKSQQEMDSSFIGLTGGQIFHEMMRRHDVDTVFGYPGGAILPVYDAIHESDAFNFILPRHEQGAGHMAEGYARATGRPGVVLVTSGPGATNVVTPMTDALADGVPLIVFTGQVATSAIGTDAFQEADVVGISRSCTKWNVMVKNVEELPKRINEAFEIAMSGRPGPVLVDLPKDVTAAVLRNPIPTKSTLPSDSLAQLTKRAQDEFTSNNVKRAADLVNVAKKPILYVGAGIFNNPDGPRLLKELSERAQIPVTTTLQGLGAFDQEDTKSLDMLGMHGCATANLAIQNADLILAVGARFDDRVTCNISKFAPEARRAALEGRGGIVHFEMTPKNISKVVETQVAVHGDAATNIQKLIPLVFGVKERQEWFNQISQWKKEYPLDYMHETPGSKIKPQTVISKLSDIANATGKEVIVTTGVGQHQMWAAQHWTWKTPRSFITSGGLGTMGYGLPAAIGAQIAKPNALVFDIDGDASFNMTLTELSSAVQAKTPIKILVLNNEEQGMVTQWQSLFYEHRYSHTHQLNPDFVKLSEAMGLKGMRLTDQSKTDEVLKEFVEYNDGPVLLEVEVAPKVPVLPMVPGGKGLEEFINYDPEMEEEQNRLRYERTSGKY

SEQ ID NO: 32 - protein, 685 aa, Zygosaccharomyces bailii
MIRQATVKQMGATRCAVRSAVRTTTRNFACYTVRYHSTTAPKQYSAATAGAKRPEPAPSFNMEPTGTPSKLHQKLKNNEQQMDSSFIGLTGGQIFHEMMRRHDVDTVFGYPGGAILPVYDAIHDSEAFNFVLPRHEQGAGHMAEGYARATGKPGIVLVTSGPGATNVVTPMTDALADGVPLIVFTGQVPTSAIGTDAFQEADVVGISRSCTKWNVMVKSVDELPRRINEAFEIAMSGRPGPVLVDLPKDVTAAVLRNPIPTKSTLPSDSLAQLTKGAHDEFTSNNVKRAADLINVAKKPILYVGGGIFNNPDGPRLIRELSERAQIPVTTTLQGLGAFDQEDVKSLDMLGMHGCATANLAVQNADLIIAVGARFDDRVTCNISKFAPEARRAALENRGGIIHFEMSPKNISKVVETQVAVHGDAATNLEKLIPLVFGVKERKQWFDQIARWKEEYPLDYMHETPGSKIKPQTVIAKLSKIANSLGKPVIVTTGVGQHQMWAAQHWTWKSPRTFITSGGLGTMGYGLPAAIGAQIARPDAIVFDIDGDASFNMSLTEMSSAVQAKAPIKIIILNNEEQGMVTQWQSLFYEHRYSHTHQLNPDFVKLAEAMGMKGMRLSDQSKTDEVLKEFVDYSEGPVLLEVEVATKVPVLPMVPGGKGLEEFINYDPEMEKEQNRLRYERTKGKY

SEQ ID NO: 33 - protein, 691 aa, Kluyveromyces lactis
MLSQRFVTRALARRAFGKTCLKLGSASASASNAASAAAGEASSRPQPAPSFNADIAGGSSGGARRNQIPLSKRSRIADKNTGVNIQNQMDDSFIGMTGGEIFHEMMQRHNVDTVFGYPGGAILPVYDAIYNSDKFTFVLPKHEQGAGHMAEGYARASGKPGVVLVTSGPGATNVVTPMADALADGVPLVVFTGQVATSAIGTDAFQEADVVGISRSCTKWNVMVKTIEELPRRINEAFEIATSGRPGPVLVDLPKDVTAAVLRNPIPMKSILPSDTLNQLTQRVVDEFTLENISRAADLINRAKKPLLYVGAGILGSEMGPQYLKQLSDRAQIPVTTTLQGLGAFNQEDHKSLDMVGMHGNAAANLAIQNADLIIALGARFDDRVTLNISKFAPEARKAALEKRGGIVHFEISPKNINKVVEAEVAVEGDVTRNLEKLLPLVESVPERPEWSAQVAKWKQEYPYAYQKETPGSKIKPQTVISKLSDLVLKSGRPAIVTTGVGQHQMWAAQHWTWKSPRSFITSGGLGTMGYGLPAAIGAQVARPDAIVIDIDGDASFNMTLMELSSAVQARTPVKVLLLNNEEQGMVTQWQSLFYEHRYSHTHQLNPDFVKLAEAMGLKALRLSKQEDTDAILKEFVDYEGPILLEVQIEKKVPVLPMVPTGKGLHEFINFDPEVEKEQNELRHKRTSGKY

SEQ ID NO: 34 - protein, 700 aa, Kluyveromyces marxianus
MLAGRIASQRSARIHSIHKLYKHSRCIAMIGGARAPPIHMVPTGTRSAASAAAVSSAETSNEQPARPQPAPSFNIPSLKNRTTTNSNSKDKHGLAQMDDSFIGMTGGEIFHEMMQRHNVDTVFGYPGGAILPVYDAIYNSDKFKFVLPRHEQGAGHMAEGYARASGKPGVVLVTSGPGATNVVTPMADALADGVPLVVFTGQVATSAIGTDAFQEADVVGISRSCTKWNVMVRSIEELPRRINEAFEIATSGRPGPVLVDLPKDVTAAVLRNPIPMKSILPSDTLNQLTQRVVDEYTLQNIGRAAELINKAKKPLLYVGAGILGHSKGPQFLKQLSDRAQIPVTTTLQGLGAFDQEDPKSLDMVGMHGNAAANLAIQNADLIIALGARFDDRVTLNISKFAPEARKAALEKRGGIVHFEISPKNINKVVEAEVAVEGDVTHNLEKLLPLIETVPERPEWSAQVQEWKQKYPYSYQRETPGSKIKPQTVISKLSKLVNSTGKPAIVTTGVGQHQMWAAQQWTWKSPRTFITSGGLGTMGYGLPAAIGAQVARPDAIVIDIDGDASFNMTLMELSSAVQARTPIKILLLNNEEQGMVTQWQSLFYEHRYSHTHQLNPDFVKLAEAMGLKALRLSDQKDTDAVLKQFIDYNDGPILLEVQVEKKVPVLPMVPTGKGLHEFINFDPEEEKKQNELRHKRTNGKH

SEQ ID NO: 35 - protein, 678 aa, Lachancea thermotolerans
MMRQSAGKMMLRRGISAALARNSARIGSLRLYSASATSTQTADDQRPRPAPSFDMEPSRSVKASKLRKSEMPLEAMDDSFIGLSGGQIFHEMMRRHKVNTVFGYPGGAILAVYDAIYNSEHFNFVLPKHEQGAGHMAEGYARVSGKPGVVLVTSGPGATNVVTPMADALADGVPMVVFTGQVATSSIGTDAFQEADVIGISRSCTKWNVMVKNVEELPRRINEAFEIAVSGRPGPVLVDLPKDVTSAILRTPIPVKSTLPSNALSKITKSAAKEFTLANLQRAADLINIAKKPLIYVGGGIFGKEQGPKLLRELAERAQIPVSTTIQGLGAFDQEDPKSLDMLGMHGSAFANLSVQNADLIIALGARFDDRVTCNVAKFAPEARKAALENRGGIIHFEISPKNINKVVEAQVAVEGDVTDNLERLIPLVNNVKERKEWFAQIDAWKKEYPYDYQRETKDSKIKPQTLVARLSEVASSTGRDVIVTTGVGQHQMWAAQHWTWRHPRTFITSGGLGTMGYGLPSAIGAQIARPDALVIDIDGDASFNMTLMELSSAVQAGAPVKIVVLNNEEQGMVTQWQSLFYEHRYSHTHQLNPDFVKLAEAMGLKGMRLSKQEDMDSVLKEFVNTEGPVLLEVIVEKKVPVLPMVPAGKGLHEFINFDAEVEKEQNELRHKRTGGKH

SEQ ID NO: 36 - protein, 688 aa, Tetrapisispora phaffia
MLRSTGSRTSLNRLAKISKSVDSQWFVTATLLKRDFTSSQLSLKSSVYSRPSPAPSFNASPTLADSTNKISQSKLKELKKNPEMDSSLIGLTGGEIFHEMMKRHNVDTVFGYPGGAILPVYDAIYNSKDFNFVLPRHEQGAGHMAEGYARASGKCGVVLVTSGPGATNVITPMADALADGVPMVVFTGQVPTAAIGTDAFQEADVIGISRSCTKWNVMVKRVEDLPKCINEAFEIATSGRPGPVLVDLPKDVQASILRAPIPIKTTIPSNALKQLTRSAYTQFSEAGIKRAAELINIAKKPILYVGGGILNNPEGPRLIKELSDRAQIPVTTTLQALGAFDQDDPKSLDMLGMHGYAPANLAIQNADLIIAVGARFDDRVTLNISKFAPEARRAALENRGGIIHFDISAKNINKVVETQVPIEGDAATNLQQMIPSVFSVTERERKEWLDTINNWKAKYPYAYQKETPGSKIKPQTVIAKLSEIANSSGKPIVVTTGVGQHQMWAAQHWTWKTPRTFISSGGLGTMGYGLPAAIGAQVARPDAVVIDIDGDASFNMSLTELSSAVQACTPIKILLLNNEEQGMVTQWQSLFYKHRYSHTHQLNPDFVKIAEAMGMKGMRLAEQSKMEDTLHEFFNFDGPVLLEVVVEKKVPVLPMVPAGKGLDEFICFDPEVEKEQNELRRKRTNGKH

SEQ ID NO: 37 - protein, 710 aa, Tetrapisispora blattae
MSLSAISRTRSVLSKWSFQSTTLHNRPCATRVFTRTLAPQRKHLSTSFITQASSPKHKRSEKVKPNDLDLEFEQNKRPEPSPSFDVDPAPKLDGTAANAKNADSPDLDHTMIGMTGGEIFNEMMRRKNVDTVFGYPGGAILPVYDAIYNSENFKFVLPRHEQGAGHMAEGYARASGKPGVVLVTSGPGATNVVTPMADALADGVPMVVFTGQVPTSAIGTDAFQEADVIGISRSCTKWNVMVKSVDELPKRINEAFEIAMSGRPGPVLIDLPKDVTAAVLKNPIPVLNNSLPYTAISNINQIIQRKFLSENIQETAKLINMAKKPVIYAGAGILSNLNGPTLLKELSERSQIPVTTTLQALGAFDQQDPKSLDMLGMHGSAVANLAIQNADLIIALGGRFDDRVTGNISKFAPEARKAALEKRGGIVHFEISPKNINKVVETQIALEGDVTENLEILLPLIKSVDERPEWFAQINQWKKAYPYEYMRETPGSKIKPQTVISRLSDIANSTGKKVIVTTGVGQHQMWTAQHWTWRNPRTFITSGGLGTMGYGLPAAIGAQVAFPEALVIDIDGDASFNMTLTELSSAVQANTPIKIMLLNNEEQGMVTQWQTLFYEHRYSHTHQMNPNFQKLAEAMGLKAMKLKEYEQIDSTLKEFLEHEGPVLLEVEIEKKVPVLPMVPAGKGLDEFMYFDPKTEKEQEKIRFERTEGKH